(19) United States
(12) Patent Application Publication
(10) Pub. No.: US 2009/0048545 A1
(43) Pub. Date: Feb. 19, 2009

(54) ULTRASONIC THERAPEUTIC APPARATUS

(75) Inventors: Naoko Kajimoto, Mizuho-shi (JP); Kyousuke Kajimoto, Mizuho-shi (JP); Atsumi Ohta, Tokyo (JP); Nobuo Ogiwara, Ageo-shi (JP)

Correspondence Address: BELL, BOYD & LLOYD, LLP, P.O. Box 1135, CHICAGO, IL, 60690, US

(73) Assignee: Bio Map Co., Ltd., Mizuho-shi (JP)

(21) Appl. No.: 12/096,159

(22) PCT Filed: May 2, 2006
(86) PCT No.: PCT/JP2006/309163

§ 371 Date: Jun. 4, 2008

(30) Foreign Application Priority Data
Dec. 7, 2005 (JP) 2005-353299

(51) Int. Cl. A61N 7/00

(52) U.S. Cl. 601/2

(57) ABSTRACT

An ultrasonic therapy device imparts ultrasonic waves to a treatment area in a mouth cavity where an implant treatment has been performed to perform therapy, including: a fitting portion that is fitted and fixed to teeth near the treatment area; a housing that is provided in the fitting portion, is disposed near the treatment area, and in which an opening portion is formed on the side of the treatment area; and an oscillator that is housed in the housing; in which a linear saliva introduction member that is arranged to project outward of the fitting portion or the housing from the opening portion of the housing is provided on the fitting portion or the housing.

### CROSS REFERENCE TO RELATED APPLICATIONS

The present application claims priority to Japanese Patent Application No. 2005-353299, filed on Dec. 7, 2005, the content of which is incorporated herein by reference.

### BACKGROUND

The present application relates to an ultrasonic therapeutic apparatus that is used when performing dental treatment using an implant. A conventional method for attaching an artificial dental root comprises embedding an artificial dental root having the shape of a pin, a so-called implant, into the bone of the maxilla or mandible, and screwing in and fixing an artificial crown into a screw hole at the end portion of the implant that is projected from the bone where it is embedded. In this method, when a fixed period from embedding the implant in the bone has passed and the embedded implant has become integrated with the bone tissue, the artificial crown is attached. In this case, during the period until the implant integrates with the bone tissue, it is necessary to use a temporary denture. Until now, since there was nothing besides leaving the process to natural healing, the period until the bone tissue is formed between the implant and the bone caused inconvenience to the patient.

In order to promote the healing process following the implant treatment, an ultrasonic therapeutic apparatus has been proposed that is provided with a housing that is disposed near a portion where the implant treatment is performed in the mouth cavity, an oscillator that is disposed in an inner wall of the housing, and a pair of fixing members that is provided on the side surfaces of the housing and which are made to cover teeth of both adjacent sides from the portion where the implant treatment is performed (for example, refer to Japanese Unexamined Patent Application, First Publication No. 2004-113625). By applying ultrasonic waves to the treatment area with this ultrasonic therapeutic apparatus, it is possible to promote the integration between the implant and the bone tissue.

However, in contrast to the vibrating surface of the oscillator that imparts ultrasonic waves, which is generally a flat surface, the treatment area where the implant treatment has been performed is an indeterminate irregularity. For this reason, a gap arises between the vibrating surface of the oscillator and the treatment area, and so efficient propagation of the ultrasonic waves to the treatment area is hindered. Also, it is conceivable to form the shape of the vibrating surface of the oscillator so as to match the shape of the treatment area, but the surface shape of the treatment area changes in accordance with the curing, or the oscillator is pressed by the surrounding muscles, whereby ex-post gaps can arise, leading to a drop in the efficiency of the ultrasonic vibration propagation.

### SUMMARY

The present disclosure provides an ultrasonic therapeutic apparatus that is capable of reliably and efficiently propagating ultrasonic waves to a treatment area where implant treatment has been performed.

In one embodiment, an ultrasonic therapy device is provided that imparts ultrasonic waves to a treatment area in a mouth cavity where an implant treatment has been performed and thereby provides therapy, including a fitting portion that is fitted and fixed to teeth near the treatment area. At least one housing that is provided in the fitting portion, is disposed near the treatment area, and in which an opening portion is formed on the side of the treatment area. An oscillator that is housed in the housing, in which a linear saliva introduction member that is arranged to project outward of the fitting portion or the housing from the opening portion of the housing is provided on the fitting portion or the housing.

According to the ultrasonic therapy device of the embodiment, as a result of the fitting portion being fitted and fixed to the teeth of a patient, the oscillator that is housed in the housing is abutted on the treatment area via the saliva introduction member. The saliva introduction member is interpositioned between the oscillator and the treatment area, and is arranged protruding outward.

Then, the linear saliva introduction member that projects outward suctions nearby saliva in the mouth cavity by the capillary phenomenon, and the suctioned saliva is filled in the gap between the oscillator and the treatment area. For this reason, the ultrasonic waves of the oscillator are reliably and efficiently propagated via the saliva that is filled in the gap. Also, the saliva is a liquid that has viscosity and is in a state of being constantly supplied to the gap between the oscillator and the treatment area by the saliva introduction member. For this reason, even when the gap between the treatment area and the oscillator changes, the saliva deforms in response to that and is supplied so that it is possible to always maintain the filled state, and it is possible to reliably and efficiently propagate the ultrasonic waves by the oscillator.

Also, in the aforementioned ultrasonic therapy device, it is more preferred that a reinforcing member be provided at a portion of the fitting portion near the treatment area.

According to the ultrasonic therapy device of the embodiment, when a reinforcing member is arranged at the portion of the fitting portion that is near the treatment area, it is possible to increase the strength of the fitting portion. Moreover, by being arranged at a portion near the treatment area, it is possible to prevent deformation and slippage of the fitting portion, and so the oscillator is reliably disposed at the treatment area, and it is possible to make the ultrasonic waves propagate.

Also, in the aforementioned ultrasonic therapy device, it is more preferred that the housing be rotatably coupled to the fitting portion.

According to the ultrasonic therapy device of the embodiment, since the housing is rotatably coupled to the fitting portion, it is possible to adjust the position of the housed oscillator with respect to the treatment area, and so it is possible to make the ultrasonic waves of the oscillator propagate to the optimal portion. Also, by successively shifting the position, it is possible to make the ultrasonic waves of the oscillator propagate over a wide range of the treatment area.

According to the ultrasonic therapy device of the embodiment, since saliva, which has viscosity, is supplied and filled into the gap between the oscillator and the treatment area by the saliva introduction member, it is possible to reliably and efficiently propagate the ultrasonic waves of the oscillator. For this reason, it is possible to reliably promote generation of bone tissue around the treatment area where the implant is performed, and possible to shorten treatment time.

Additional features and advantages are described herein, and will be apparent from, the following Detailed Description and the figures.

### BRIEF DESCRIPTION OF THE FIGURES

FIG. 1 A is a perspective view of the ultrasonic therapy device of the first embodiment.

FIG. 1B is a descriptive view in which the ultrasonic therapy device of the first embodiment is mounted in the mouth cavity.

FIG. 2 is a perspective view in which a part of FIG. 1A and FIG. 1B of the ultrasonic therapy device of the first embodiment is cross-sectionally shown.

FIG. 3 is a perspective view of the ultrasonic therapy device of the second embodiment.

FIG. 4 is a perspective view of the ultrasonic therapy device of the third embodiment.

### DETAILED DESCRIPTION

### First Embodiment

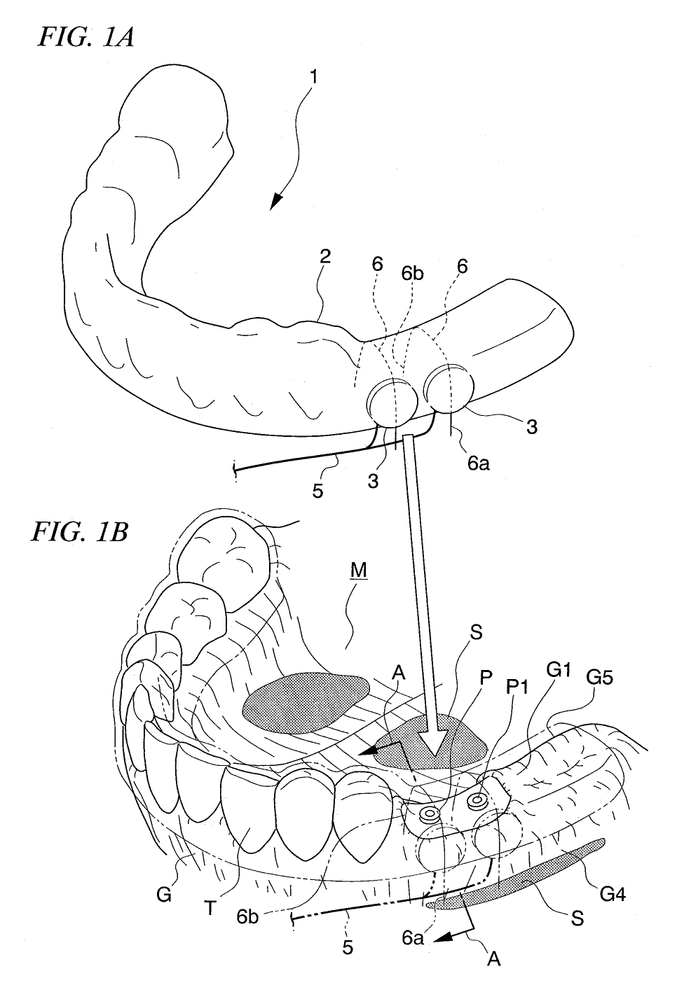

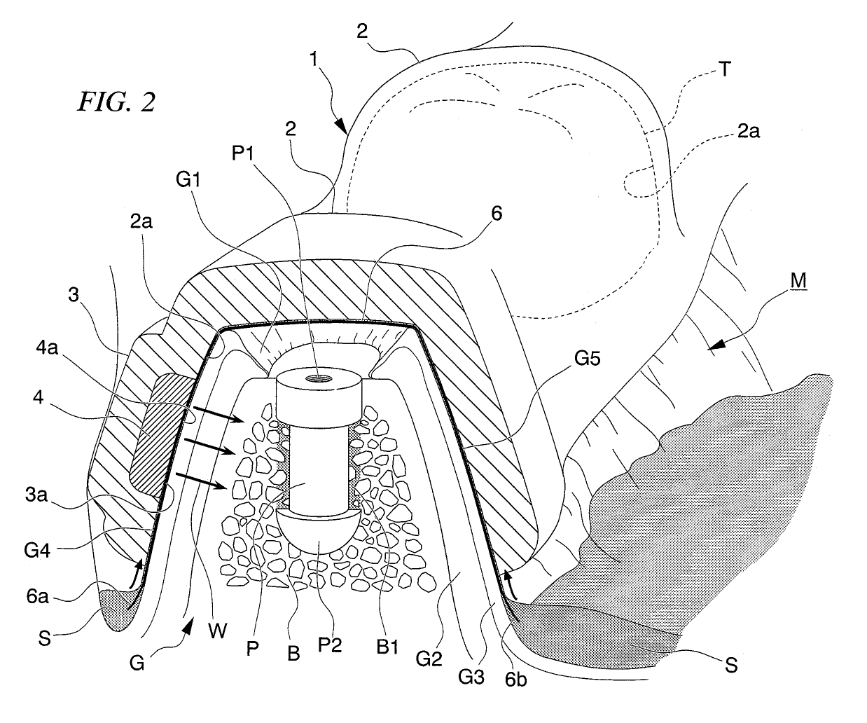

FIG. 1A to FIG. 3 shows a first embodiment. FIG. 1A shows an overall perspective view of an ultrasonic therapy device. FIG. 1B is an explanatory view in which the ultrasonic therapy device of FIG. 1A is mounted in a mouth cavity. As shown in FIG. 1B, in the mouth cavity M, a tooth T is partially missing, the gum G is exposed, and a treatment area G1 where implant treatment has been performed is formed. In greater detail, at the treatment area G1, an implant P is embedded as an artificial dental root in the mandible, and a screw hole P1 to which an artificial crown is to be screwed in is provided on the upper face of the implant P. Moreover, FIG. 2 shows a partially cross-sectional perspective view along section A-A of FIG. 1B. As shown in FIG. 2, the implant P is embedded in bone B of the mandible at a base end portion P2, and fixed by generated bone tissue B1. That which is covered around the bone B is the gum G, which is constituted by an epidermis G3 that is wrapped on the outer side of a gingival tissue G2. Also, saliva S that is secreted from within the human body accumulates on both sides of the gum G.

As shown in FIGS. 1A, 1B and FIG. 2, an ultrasonic therapy device 1 is provided with a fitting portion 2 that is fitted and fixed to a tooth T, a housing 3 with an opening portion 3a formed on the side of the treatment area G1, and an oscillator 4 that is housed in the housing 3. An inner periphery 2a of the fitting portion 2 is formed to fit the shape of the tooth T and the gum G of the patient, and the fitting portion 2 is fitted and fixed by being closely attached to the tooth T. Moreover, at the treatment area G1, the inner periphery 2a of the fitting portion 2 is formed in conformance with the shape of the gum G of the patient, and fitted and fixed by being closely attached to the gum G. Also, the housing 3 is provided in the fitting portion 2 so as to be at a position near the treatment area G1 on the side of an outer periphery G4 of the gum G. Also, the oscillator 4 has an oscillating surface 4a that abuts and propagates ultrasonic waves, with a lead wire 5 connected thereto and connected to an ultrasonic signal supply source outside the mouth cavity which is not illustrated.

Also, as shown in FIGS. 1A, 1B and FIG. 2, the ultrasonic therapy device 1 has a linear saliva introduction member 6 provided on each housing 3. The saliva introduction member 6 is fixed to the fitting portion 2 and the housing 3 at the position corresponding to the treatment area G1, and one end 6a is disposed projecting outward of the housing 3 from the opening portion 3a of the housing 3. Also, another end 6b is disposed projecting outward of the fitting portion 2 at the side of an inner periphery G5 of the gum G via the inner periphery 2a of the fitting portion 2 from the opening portion 3a of the housing 3.

Next, the action of the ultrasonic therapy device 1 shall be described. As shown in FIG. 1B and FIG. 2, the ultrasonic therapy device 1 is mounted in the mouth cavity M, and the fitting portion 2 is fitted and fixed to the tooth T and the gum G. For this reason, the oscillator 4 that is housed in the housing 3 is abutted on the side of the outer periphery G4 of the treatment area G1 via the saliva introduction member 6 that is arranged in the opening portion 3a. Also, the saliva introduction member 6 is in an interpositioned state by being closely contacted between the treatment area G1 and the combination of the fitting portion 2 and oscillator 4. Then, with the one end 6a at the side of the outer periphery G4 of the gum G and the other end 6b at the side of the inner periphery G5 so as to be respectively arranged on the gum G, the deposited saliva S permeates. Since the saliva introduction member 6 is formed in a linear shape, the saliva S is suctioned by the capillary phenomenon of the saliva introduction member 6 and filled in the gap that is formed between the oscillator 4 and the treatment area G1. For this reason, the ultrasonic waves W of the oscillator 4 are reliably and efficiently propagated from the oscillator 4 directly to the treatment area G1 or to the treatment area G1 via the saliva S that is filled in the gap.

Also, the saliva S has viscosity, is always supplied in the mouth cavity M, and maintains a state of a definite quantity being accumulated. That is, even if the gap between the oscillator 4 and the treatment area G1 changes, the saliva S that is filled therein changes shape due to its own viscosity in a manner corresponding to that gap, with the saliva S being supplied via the saliva introduction member 6 to portions where a shortage of the saliva S arises so that it is possible to maintain the filled state. For example, in the case of the surface shape of the treatment area G1 changing as the curing proceeds, or even if a gap arises as a result of the oscillator 4 being pressed by the surrounding muscle, in response to that gap, the saliva S deforms or is supplied to maintain the filled state. For this reason, the ultrasonic waves W of the oscillator 4 are always accurately and efficiently propagated, and so it is possible to reliably promote generation of bone tissue B1 around the treatment area G1 where the implant P is performed, and so the treatment time can be shortened.

Note that the material of the saliva introduction member 6 may be one formed by various materials including metal such as stainless steel or aluminum, resin such as acrylic or nylon, and silicon, and may be one having flexibility or one having rigidity. In the case of a rigid material, the saliva introduction member 6 is formed in advance to fit the shape of the treatment area G1 to be abutted and preferably has a shape that is capable of coming into close contact with the treatment area G1. Also, the saliva introduction member 6 may be formed in a fibrous shape, a sponge shape, or a tubular shape, whereby it is possible to expect the suction effect of the saliva S by the capillary phenomenon. Also, the saliva S is filled in the gap between the oscillator 4 and the treatment area G1, but a gel may be interposed between the oscillator 4 and the treatment area G1 in advance. By doing so, it is possible to more reliably and efficiently propagate the ultrasonic waves W of the oscillator 4 in combination with the effect by filling the space with the saliva S. Note that in the case of interposing a gel in this manner, when an excess of the gel results from a change in the surface shape of the treatment area G1, the saliva introduction member 6 causes the gel to flow back to the mouth cavity M by the capillary phenomenon, and so the gel amount is adjusted to the proper amount.

### Second Embodiment

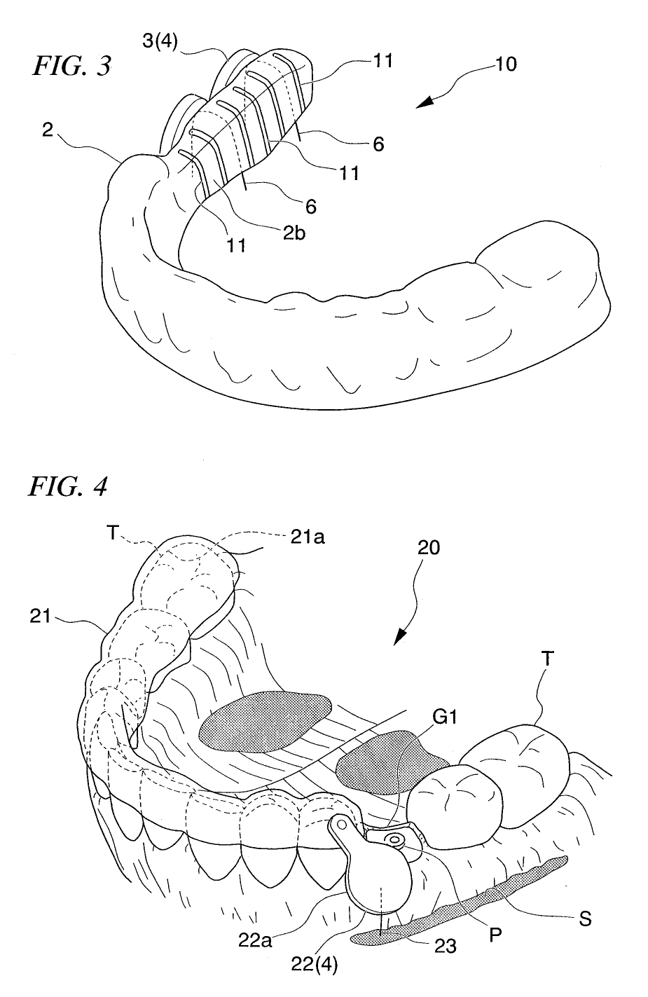

FIG. 3 shows a second embodiment according, being an overall perspective view of the ultrasonic therapy device. In this embodiment, the same reference numerals shall be assigned to members that are the same as those used in the above embodiment, and descriptions thereof shall be omitted.

As shown in FIG. 3, in an ultrasonic therapy device 10 of the present embodiment, at the portion of the fitting portion 2 near the treatment area G1, reinforcing members 11 are arranged on the outer periphery 2b of the fitting portion 2. The reinforcing members 11 are formed for example from metal such as stainless steel and aluminum or resin and the like. According to this ultrasonic therapy device 10, it is possible to increase the strength of the fitting portion 2 with the reinforcing members 11, and thereby enable use over a long period. Also, it is possible to prevent deformation and slippage of the fitting portion 2 with the reinforcing members 11. For this reason, the oscillator 4 that is housed in the housing 3 is reliably disposed at the treatment area and, in conjunction with the effect due to the aforementioned saliva introduction member 6, it is possible to reliably and efficiently propagate ultrasonic waves of the oscillator 4 to the treatment area. Note that the reinforcing member 11 is not limited to the linear member as shown in FIG. 3, and may also be a plate-shaped member or the like.

### Third Embodiment

FIG. 4 shows a third embodiment, being an overall perspective view of the ultrasonic therapy device. In this embodiment, the same reference numerals shall be assigned to members that are the same as those used in the above embodiment, and descriptions thereof shall be omitted.

As shown in FIG. 4, an ultrasonic therapy device 20 of the present embodiment is provided with a fitting portion 21 that is fitted and fixed to a portion of a tooth T and a housing 22 that houses the oscillator 4 and is coupled in a rotatable manner to the fitting portion 21. An inner periphery 21a of the fitting portion 21 is formed to match the shape of the teeth T, and the fitting portion 21 is formed for example with a resin or the like that is hard and does not deform. In a housing 22, an opening portion 22a is formed on the side of the treatment area G1. Also, the ultrasonic therapy device 20 is further provided with a saliva introduction member 23 that is fixed to the housing 22 and arranged from the opening portion 22a of the housing 22 to the outside of the housing 22. The saliva introduction member 23 is formed with the same materials as stated above.

According to this ultrasonic therapy device 20, the housing 22 is capable of rotating with respect to the fitting portion 21, and so by performing position adjustment of the housed oscillator 4 with respect to the position portion G1, it is possible to make the ultrasonic waves propagate to the optimal portion. Also, by successively shifting the position, it is possible to make the ultrasonic waves propagate over a wide range of the treatment area G1. Moreover, since the housing 22 is coupled to the fitting portion 21 in a cantilevered state at the center of rotation, the housing 22 is pressed by the mouth wall, and so the housed oscillator 4 is made to closely contact the treatment area G1. For this reason, in conjunction with the effect due to the saliva introduction member 23, it is possible to reliably propagate the ultrasonic waves W of the oscillator 4 to the treatment area G1, and reliably promote generation of bone tissue B1 around the treatment area G1 that is fitted with the implant P, and so the treatment time can be shortened. Note that in the present embodiment, the saliva introduction member 23 projects only to the side of the outer periphery, but even when supplying the saliva S by being thus projected only to one side, the gap between the treatment area G1 and the oscillator is filled by the saliva S, and so the same effect is obtained.

Preferred embodiments have been described in detail above with reference to the drawings, but specific constitutions are not limited to these embodiments, and modifications can be made without departing from the spirit or scope of the present invention.

Note that the ultrasonic therapy device in each embodiment was provided on the mandible portion, but the same effect can be obtained even when used for therapy on an implant treatment area on the maxilla portion by being provided on the maxilla portion. Also, it is not necessary for the fitting portion to be fitted on the entire maxilla portion or the mandible portion. It may at the least be fitted on some teeth T that are near the treatment area G1 provided it is possible to fix the housing to the predetermined position. A mode is also possible in which a U-shaped wire is fitted on the teeth. Moreover, the housing and the oscillator that is housed in the housing were described as being provided on the side of the outer periphery of the treatment area G1. However, they are not limited thereto and may be provided on the side of the inner periphery G5 of the treatment area G1 depending on the situation of the treatment area G1, and may be provided on both the side of the outer periphery G4 and the side of the inner periphery G5. Also, two pairs of the housing and the oscillator were provided in the first embodiment and the second embodiment, while one pair was provided in the third embodiment. However, the embodiments are not limited thereto, such that three pairs or more of the housing and the oscillator may be provided.

Moreover, one corresponding saliva introduction member was disclosed as being provided for each housing, but a plurality may also be provided.

Next, the effects of using the ultrasonic therapy devices 1, 10, 20 of the above explained embodiments shall be explained.

1) Accelerating osteogenesis and osteointegration around an affected portion of an implant fixture after embedding the implant fixture. 2) Accelerating osteogenesis and osteointegration around an affected portion of an implant fixture after removing the implant fixture. 3) Accelerating ostegenesis in dentary defect around an implant fixture. 4) Accelerating ostegenesis in dentary hyperplasia for a donor and a recipient.

5) Accelerating ostegenesis in a technique for extension osteogenesis. 6) Accelerating postoperative healing in a maxillofacial injuries, maxillofacial fractures, and tooth dislocations. 7) Accelerating ostegenesis after surgery to a maxillofacial deformity. 8) Minimizing a non-retention period and a retention period during orthodontic therapy. 9) Accelerating ostegenesis in an osteorrhaphy portion after a redressment. 10) Accelerating a secondary dentine organization after a vital pulp cutting and direct or indirect vital pulp covering in a repair of dental caries. 11) Pain reduction for a dentine hyperesthesia by accelerating a secondary dentine organization. 12) Accelerating blocking of an apical area after a removal of a vital pulp and an operation for an inflected vital pulp. 13) Accelerating recovery after a periodontal surgery (Flap operation FGG APF). 14) Accelerating osteogenesis after a tooth fixing in an occlusal trauma. 15) Accelerating recovery for insufficient healing after surgery.

16) Accelerating recovery of abnormal frenulum after surgery. 17) Accelerating recovery after a surgery for a cheiloschisis, a palatoschisis, and a cheilognathoschisis. 18) Accelerating osteogenesis after a surgery of ankylosis of the temporomandibular joint. 19) Accelerating osteogenesis and recovery after surgery for osteitis and osteomyelitis. 20) Accelerating a normalization for a function of sialaden. 21) Accelerating bone healing after an osseous nidus exenteration in an odontological maxillofacial area. 22) Accelerating osteogenesis in a periodontal osseous defect (GTR and GBR technique). 23) Accelerating osteoatresia of a root apex during dental therapy (including APICO).

24) Accelerating healing of a wound after removing a soft tissue in an odontological maxillofacial area, a soft tissue damaged by trauma, and a surgically modified soft tissue. 25) Accelerating osteogenesis of new tissue around an implanted tooth and a re-planted tooth. 26) Accelerating curing of an exodontias cavity. 27) Relieving muscle fatigue in temporomandibular arthrosis. 28) Accelerating osteogenesis in glenmoid head and clenoid cavity in temporomandibular arthrosis. 29) Accelerating healing of a surface lesion in soft tissue.

With the ultrasonic therapy device according to the present embodiment, the fitting portion is fitted and fixed to the teeth of a patient, and the oscillator that is housed in the housing is made to abut a treatment area via a saliva introduction member. The saliva introduction member is interpositioned between the oscillator and the treatment area, arranged projecting outward, and suctions nearby saliva in the mouth cavity due to the capillary phenomenon, with the suctioned saliva being filled in a gap that is formed between the oscillator and the treatment area. For this reason, the ultrasonic waves of the oscillator are reliably and efficiently propagated via the saliva that is filled in the gap. Also, since the saliva is a liquid that has viscosity and is in a state of being constantly supplied to the gap between the oscillator and the treatment area by the saliva introduction member, even when the gap between the treatment area and the oscillator changes, the saliva deforms in response to that and is supplied so that it is possible to always maintain the filled state, and it is possible to reliably and efficiently propagate the ultrasonic waves by the oscillator.

Furthermore, when a reinforcing member is arranged at the portion of the fitting portion that is near the treatment area, it is possible to increase the strength of the fitting portion. Moreover, it is possible to prevent deformation and slippage of the fitting portion, and so the oscillator is reliably disposed at the treatment area, and it is possible to cause the propagation of the ultrasonic waves.

Also, since the housing is rotatably coupled to the fitting portion, it is possible to adjust the position of the housed oscillator with respect to the treatment area, and so it is possible to make the ultrasonic waves of the oscillator propagate to the optimal portion. Also, by successively shifting the position, it is possible to make the ultrasonic waves of the oscillator propagate over a wide range of the treatment area.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1-3. (canceled)

4: An ultrasonic therapy device configured to provide therapy by imparting ultrasonic waves to a treatment area in a mouth cavity where an implant treatment has been performed, the ultrasonic therapy device comprising:
a fitting portion that is fitted and fixed to teeth near the treatment area;
at least one housing that is provided in the fitting portion, is disposed near the treatment area, and in which an opening portion is formed on the side of the treatment area; and
an oscillator housed in the housing;
wherein a linear saliva introduction member that is arranged to project outward of the fitting portion or the housing from the opening portion of the housing is provided on the fitting portion or the housing.

5: An ultrasonic therapy device according to claim 4, wherein a reinforcing member is provided at a portion of the fitting portion near the treatment area.

6: An ultrasonic therapy device according to claim 4 or claim 5, wherein the housing is rotatably coupled to the fitting portion.
